(19) United States
(12) Patent Application Publication
(10) Pub. No.: US 2004/0158407 A1
(43) Pub. Date: Aug. 12, 2004

(54) "Recurrent signature" identifies transcriptional modules

(76) Inventors: Jan Ihmels, Rehovot (IL); Sven Bergmann, Tel Aviv (IL); Naama Barkai, Rehovot (IL)

Correspondence Address: Anthony Castorina, GE Ehrlich, Suite 207, 2001 Jefferson Davis Highway, Arlington, VA, 22202, US

(21) Appl. No.: 10/471,575

(22) PCT Filed: Mar. 14, 2002
(86) PCT No.: PCT/IL02/00211

(30) Foreign Application Priority Data
Mar. 14, 2001 (IL) 142006

(51) Int. Cl. G01N 33/48

(52) U.S. Cl. 702/19

(57) ABSTRACT

A method for analyzing and identifying functional linkages between biological processes which are regulated in a coordinate manner. An especially preferred example of a biological process to which the method of the present invention may be applied is the analysis of groups of coordinately regulated genes. More preferably, the present invention analyzes large set of genome-wide expression data or transcriptional data in order to discover coordinately regulated genes, which can be termed “transcriptional modules”. Optionally and most preferably, the groups of genes are identified with their corresponding cis-regulatory elements. Thus, if the cis-regulatory elements for the transcriptional module are not known, they can optionally be identified with the method of the present invention, for example if functional linkages between genes are known and the associated cis-regulatory element is also available, this information can be used to identify the corresponding transcriptional module.

### FIELD OF THE INVENTION

The present invention is of a method for determining functional and/or other biologically relevant linkages between different biological processes, and in particular, of such a method which is useful for determining such linkages between genes which are regulated in a coordinate manner.

### BACKGROUND OF THE INVENTION

Recent advances in genomic techniques have led to the accumulation of a wealth of experimental data, which in many organisms includes genome wide sequence information and large-scale gene expression data. The extent of available data is rapidly growing, stressing the need for new computational tools that are able to extract biological knowledge from this information. However, many such computational tools for analysis of genomic data are intended for the management and initial analysis of large amounts of raw data. These tools therefore cannot uncover more functional or biological information from the raw data, which would require computational tools that are conceptually different.

One example of the type of functional, biological information which would be interesting to analyze is the regulation of gene expression. Adaptation of cells to different environments occurs through extensive changes in gene expression. Similar changes accompany most cellular processes including cell cycle progression and signal transduction. Although many gene components affecting transcription have been identified, the principles underlying the genome-wide expression program remain to be elucidated. Identifying groups of genes that are subject to similar transcriptional control is an essential first step in characterizing the transcriptional regulatory network (1).

Commonly used computational methods for clustering gene expression data tend to group genes into discrete modules that do not overlap (2, 3, 4, 5). Gene expression, however, is usually controlled by the integrated effect of several transcription factors, suggesting that genes may be part of more than one transcriptional module (6, 7, 8). Such combinatorial regulation further implies that transcriptional modules are defined with respect to a particular cellular context, and exhibit a coordinated expression change only in a relevant subset of experimental conditions. The identification of this relevant subset poses an important computational challenge (9, 10).

Although both genes and experiments may belong to several functional categories and should thus be included in multiple clusters, commonly used methods classify genes and experiments into mutually exclusive clusters. Secondly, while groups of genes may be co-regulated only under a subset of conditions, clustering methods measure similarity between genes using all conditions, and similarity between conditions using all genes. Avoiding this problem by focusing the analysis on an a priori specified subset of experiments significantly reduces the dataset and precludes the identification of novel regulatory contexts. Ideally, therefore, one would like to co-classify genes and conditions by considering the expression coherence of all subsets of genes over all combinations of experiments. This is clearly computationally infeasible even for a moderately sized dataset.

The ability to focus the analysis on the subset of relevant conditions should enable the identification of overlapping modules and eliminate the noise added by the non-relevant experiments. Information obtained from experiments in which a specific transcriptional module was co-regulated may provide further insights into the biological function and regulation of this module.

### SUMMARY OF THE INVENTION

The background art does not teach or suggest a method for analyzing information about biological processes in order to discover linkages between these processes. In particular, the background art does not teach or suggest such a method which is useful for uncovering coordinately regulated groups of biological processes. Furthermore, the background art does not teach or suggest such a method which is capable of analyzing large amounts of genomic data in order to identify groups of coordinately regulated genes, nor does the background art teach or suggest such a method which is also useful for identifying the corresponding cis-regulatory elements for these groups of genes.

There is therefore an unmet need for, and it would be useful to have, a method for analyzing large amounts of data regarding biological processes which are regulated in a coordinate manner, particularly with regard to the functional behavior of groups of genes for identifying coordinately regulated genes, preferably with regard to genes which are regulated at the level of gene expression, and also optionally to identify the corresponding cis-regulatory elements.

The present invention provides these desired features through a method for analyzing and identifying functional linkages between biological processes which are regulated in a coordinate manner. An especially preferred example of a biological process to which the method of the present invention may be applied is the analysis of groups of coordinately regulated genes. More preferably, the present invention analyzes the expression or transcriptional data obtained from the observation of groups of genes in order to discover coordinately regulated genes, which can be termed “transcriptional modules”. Optionally and most preferably, the groups of genes are identified with their corresponding cis-regulatory elements. Thus, for example if functional linkages between the genes are known, and the corresponding cis-regulatory element is also known, this information can be used to identify the transcriptional modules.

However, even if this basic information is not available, the method of the present invention is still operative in order to identify the transcriptional module for a group of genes, as well as the cis-regulatory elements. For example, the method of the present invention is also operative to identify the transcriptional module and the cis-regulatory elements even if only the gene expression data and the genomic sequence are known.

According to optional but preferred embodiments of the present invention, the method of the present invention is useful for identifying novel cis-regulatory elements themselves, as well as the genes regulated by these elements. The method of the present invention is also optionally useful for the functional assignment of uncharacterized open reading frames.

The method of the present invention has a number of advantages over methods known in the background art. For example, the method is preferably used for identifying overlapping groups of co-regulated genes, and context-dependent regulation of these genes, which are two issues that are not handled well by the background art, if they are handled at all.

According to the present invention, there is provided a method for analyzing experimental data obtained from observing biological processes which are regulated in a coordinate manner, comprising: analyzing the experimental data to identify at least two biological processes undergoing a significant, recurrent change under similar experimental conditions; and identifying functional linkages between the biological processes according to said analysis of the experimental data.

According to this preferred embodiment of the method of the present invention, if these two or more biological processes show changes in their behavior under similar experimental conditions, which may optionally be found in the same experiment for example, and more preferably if this change is recurrent, then the processes may be functionally linked. By “recurrent”, it is more preferably meant that the change occurs more than once and that the detection of such changes is statistically significant.

The present invention may optionally be generalized to such analysis of biological processes according to a preferred embodiment of the present invention as follows. First, data is received from a plurality of experimental results. Next, the experimental results are scored according to a change in a behavior of a biological process. Next, each biological process is scored according to a change in the experimental results having at least a minimum score. The biological process is considered to be regulated in a coordinate manner with at least one other biological process if the biological processes receive a score above a minimum level.

Optionally, the biological process is performed by a gene, and the experimental results measure a change in expression of the gene. Preferably, the minimum level is determined according to a statistically significant difference.

It should be noted that the method of the present invention is preferably performed by a computer, as described in greater detail below, such that the present invention may optionally and preferably be realized as computerized code (software program) which is operative for performing the method of the present invention.

### BRIEF DESCRIPTION OF THE DRAWINGS

The invention is herein described, by way of example only, with reference to the accompanying drawings, wherein:

FIG. 1 demonstrates the Recurrent Signature method of the present invention. (a) The method of the present invention receives as an input a set of genes. Gene expression data is used to identify the subset of experiments in which the input set is co-regulated (experimental signature), and subsequently the module of co-regulated genes (gene signature). (b) Recurrence property. When the input sets include different subsets of genes belonging to the same regulatory module, distinct input sets may define essentially identical modules. This Recurrent property is a key factor in distinguishing the true co-regulated modules. (c) In a general, exemplary application of the method of the present invention, the signature algorithm is applied to different input sets. Transcriptional modules are obtained by fusing recurrent signatures.

FIGS. 1D and 1E describe different input sets used for performing the method of the present invention.

FIG. 2 graphically illustrates results which use the method of the present invention to identify the amino-acid biosynthesis module. The inset of (a) schematizes the high overlaps between the computational (I and II) and experimental (15) (III) modules. The two computational modules are essentially identical, with 119 common genes (out of a total of 126 and 144). (a) The computational score of each gene is plotted against the fold repression in the GCN4-deletion strain. The gene score measures the degree of co-regulation with the module, and was obtained by applying the method of the present invention to the MIPS-classified input set. The horizontal line denotes the threshold above which a gene is assigned to the computational module, while the vertical line denotes the 2-fold repression, above which the experimental change is considered significant by convention. Forty-six genes were identified by both methods. Only five experimentally repressed genes are not included in the computational module. Note that many of the ninety-eight genes that were assigned only to the computational module were in fact repressed experimentally. (b) Different thresholds for determining the experimentally significant repression level were considered. The plot depicts the number of significantly repressed genes (left axis, designated as “black”), and the percentage of them that were identified computationally (right axis, designated as “red”), as a function of the significance threshold. Note that the computational module includes most of genes that were repressed by at least 1.5 fold. Below this threshold, the number repressed genes increases dramatically, possibly reflecting the experimental noise.

FIG. 3 demonstrates the Recurrence property of the method of the present invention. (A) The signature algorithm was applied to all heptamer-associated signatures as input sets (c.f. text). Shown is the probability that the overlap between two signatures is higher than some threshold using either real or randomized expression data. The overlap was computed both for the signatures obtained from input sets corresponding to all pairs of sequences (indicated by triangles) and to sequences differing only by one nucleotide (squares). The probability to exceed a certain overlap is consistently higher in the case of real expression data. Using only input sets corresponding to ‘neighboring’ sequences results in a further increase in the probability. (B) Multiple input sets were generated by doubling the size of a gene set through the addition of arbitrary genes. Shown is the measured probability that the overlap between the resulting signatures exceeds a certain threshold both for sets composed of co-regulated genes (squares) and for the sets composed of random genes (triangles).

FIG. 4 Co-regulation properties of the genes involved in the TCA-cycle. (A) An input set containing the yeast genes homologous to the E. coli TCA cycle genes was defined using a standard BLAST search. The recurrent signature method was applied to this input set and yielded only genes that are indeed involved in the TCA cycle. (B-C) Two subparts of the TCA cycle were found to be autonomously co-regulated. In each figure, the TCA cycle genes that were assigned to the modules are highlighted and additional genes are indicated. Note that each module is co-regulated under different sets of conditions, as indicated. The different deletions in (B) include three genes that are involved in mitochondria function (Ymr293c, Aep1 and Yer050c).

FIG. 5 shows higher order correlations between modules can be identified comparing the scores of the experiments included in both modules (see Table 1 for information). (A) In order to identify such correlations the experiment signature of a reference module is shown (vertical axis) and the scores allotted to these experiments in the other modules are plotted (horizontal axis). The number of lines indicates the number of experiments that each module shares with the reference module, while the scores of these experiments are represented by color-coding. (B+C) Two specific pairs of modules are highlighted. Note, for example, that most conditions that induce the mating genes (module #6), repress the genes that are involved in the G1/S transition during the cell cycle (module #13), reflecting the G1 arrest accompanying the mating response.

FIG. 6 illustrates the functional coherence of the modules for a selection of modules (see Table 1 for information). The genes in each module were ordered according to their score and annotated using the information provided by the YPD. While this work was in progress, the first gene of module #10 (Sda1, annotated here as unrelated) was found to be part of the 60S pre-ribosomal particle. Three other genes in the same module (Ygr103w, Yor206w, and Ylr002c, all annotated here as unknown) were shown to be involved in rRNA synthesis.

FIG. 7 graphically illustrates the identification of regulatory modules in a model-network according to the method of the present invention. Computer generated data was analyzed by (a) a hierarchical clustering algorithm or (b) the Recurrent Signature method. (a) A computational module was defined by the union of clusters from a particular tree level, which include the largest number of genes belonging to a specific underlying module. The overlap (OL) between this computational module and the underlying module is shown as a function of the number of clusters composing the computational module. A single cluster is relatively homogenous but includes only a small fraction (˜20%) of the genes in the underlying module. This fraction is denoted by the efficiency. The overlap remains small for increasing number of clusters, reflecting the fact that the large majority of the additional genes do not belong to a single underlying transcriptional module. Here a typical gene is regulated by four transcription factors. (b) The capacity of the Recurrent Method of the present invention to identify the underlying transcriptional modules is shown as a function of the typical number of transcription factors regulating a gene. Note that all computationally identified modules are identical to an underlying module, with OL˜1. Not all modules are identified at a high level of combinatorial regulation. The fraction of modules that are identified (frac.) increases with the number of input sets, as is shown in the inset for the case in which a typical gene is regulated by four transcription factors. Input sets were generated by clustering randomly chosen subsets of conditions.

### BRIEF DESCRIPTION OF THE TABLES

The invention is herein described, by way of example only, with reference to the accompanying tables, placed at the end of the text, wherein:

Table 1: Properties of a selection of modules. The module description includes the number of genes, number of recurrent signatures defining the module and the highest overlap between them. The top five genes and conditions are specified. Repressing conditions are denoted by (−). For each consensus motif a graphical representation of its nucleotide composition is shown together with the name (when known), positional bias⁹ (PBias) and number of motif appearances (MApp) within the module as compared to a random control group of genes (shown in brackets).

Table 2: Putative cis-regulatory elements identified by neighbor analysis. Shown are the number of sequence variations (# Seq.), overlap between modules (OL), number of genes in the module (Size), number of consensus sites found in the upstream regions of the genes inside the module (# Sites) compared to the average number of sites found in a random set of genes of the same size (in parentheses), and its positional bias inside the module (Pos. Bias, (21, 22)). For all motifs shown in Table 2, the positional bias for randomly chosen groups of genes is ˜10⁻². The two overlaps shown correspond to the overlap between the function-related and motif-related modules and to that between two motif-related modules identified in the neighbor analysis.

### DESCRIPTION OF THE PREFERRED EMBODIMENTS

The present invention is of a method for analyzing and identifying functional linkages between biological processes which are regulated in a coordinate manner. An especially preferred example of a biological process to which the method of the present invention may be applied is the analysis of groups of coordinately regulated genes. More preferably, the present invention analyzes large set of genome-wide expression data or transcriptional data in order to discover coordinately regulated genes, which can be termed “transcriptional modules”. Optionally and most preferably, the groups of genes are identified with their corresponding cis-regulatory elements. Thus, if the cis-regulatory elements for the transcriptional module are not known, they can optionally be identified with the method of the present invention, for example if functional linkages between genes are known and the associated cis-regulatory element is also available, this information can be used to identify the corresponding transcriptional module.

However, even if this basic information is not available, the method of the present invention is still operative in order to identify the transcriptional module, as well as the cis-regulatory elements. For example, the method of the present invention is also operative to identify the transcriptional module and the corresponding cis-regulatory elements even if only the gene expression data and the genomic sequence is available.

According to optional but preferred embodiments of the present invention, the method of the present invention is useful for identifying novel cis-regulatory elements themselves, as well as the genes regulated by these elements. The method of the present invention is also optionally useful for the functional assignment of uncharacterized open reading frames.

The present invention may optionally be described as a ‘Recurrent Signature ’ approach for identifying such transcriptional modules of coordinately expressed genes. The method preferably combines a large set of genome-wide expression data with full genome sequence information. In contrast to the background art method of clustering gene expression data, which is sufficient only when regulatory modules do not overlap, the present method can detect overlapping modules. The noise in identifying any specific module is reduced by excluding non-relevant experiments. Thus, the method of the present invention is a novel integrated method for preferably detecting cis-regulatory elements together with the associated module of co-regulated genes.

According to preferred embodiments of the present invention, a transcriptional module is more preferably specified by a two dimensional signature, which includes the co-expressed genes and the subset of relevant experimental conditions. Module signatures are optionally and preferably determined with respect to input sets of genes that comprise genes with similar functions, or genes that display a common regulatory motif in their upstream region.

The preferred algorithm of the present invention receives as input a set of genes and proceeds in two stages. First, each experiment is scored by the average expression change of the input genes. Second, each gene of the entire genome is scored by its average expression change over those experiments that received a statistically significant score. Thus, a gene is assigned a high score if its expression is induced under the same conditions as the average expression of the input genes. The output of the algorithm consists of the genes and experiments that obtained a statistically significant score. These output sets may be referred to as gene-signature and experiment-signature, respectively.

According to a specific, preferred implementation of the present invention, a signature is created for each group of genes, using large-scale gene expression data. The signature is more preferably two-dimensional. Preferably, the signature is created by first scoring each experimental condition by the average (log) fold change in the expression of the input genes at that specific experiment. The experimental conditions which induce a coordinated change in the expression of genes in the input set therefore receive a high score and comprise the experimental signature.

More specifically, the method of the present invention for the signature algorithm is preferably performed as follows. The element E_{cg} of the gene expression matrix contains the log-fold expression-change of gene g εG={1, . . . , N_{g}} at the experimental condition cεC={1, . . . , N_{c}}, where N_{g} (N_{c}) denotes the total number of genes (conditions). Two normalized expression matrices \tilde{E}^{g}_{c} Ê^{c}_{g} and Ê^{c}_{g} \tilde{E}^{g}_{c} are introduced, which have zero mean and unit variance with respect to genes and conditions, respectively:

$${⟨ {\tilde{E}}_{c}^{g} ⟩}_{g ∈ G} = 0 , {⟨ {( {\tilde{E}}_{c}^{g} )}^{2} ⟩}_{g ∈ G} = 1$$

$$and {⟨ {\hat{E}}_{g}^{c} ⟩}_{c ∈ C} = 0 , {⟨ {( {\hat{E}}_{g}^{c} )}^{2} ⟩}_{c ∈ C} = 1 ,$$

where [symbol] ... [symbol] ₓ denotes the average with respect to x. The initial input set is a collection of N₁ genes: G₁={g₁, . . . , g_{N}_{₁}}⊂G.

In the first stage of the signature algorithm all experiments are scored according to the average change in the expression within the input set. The experimental score is

$${s}_{c} = {⟨ {\tilde{E}}_{c}^{g} ⟩}_{g ∈ {G}_{l}} .$$

The experiment-signature S_{c} contains those conditions whose absolute score is statistically significant: S_{c}={cεC:|s_{c}|>t_{c}σ_{c}}. The value t_{c}=2.0 may be used as the threshold level and the standard deviation expected for random fluctuations

$${σ}_{c .} = 1 / \sqrt{{N}_{l} {N}_{g}}$$

in the present analysis.

In the second stage all genes are scored according to the weighted average change in the expression within the experiment-signature. The gene score is

$${s}_{g} = {⟨ {s}_{c} {\hat{E}}_{g}^{c} ⟩}_{c ∈ {{S}_{c}}^{.}} .$$

The gene-signature S_{g} contains those conditions whose score is statistically significant:S_{g}={gεG:s_{g}>t_{g}σ_{g}}. Here the value t_{g}=3.0 and the measured standard deviation σ_{g}=σ(s_{g}) may be used.

According to a preferred embodiment of the present invention, the method may preferably be applied to fusion of signatures in the analysis of sequence elements and pathways. The signature algorithm is applied to a reference input set Gₗ^{ref} as well as to a set of input sets {Gₗ⁽ⁱ⁾} obtained from Gₗ^{ref} by fragmentation or gene addition, resulting in the reference signature S_{ref} and a collection of modified signatures {Sᵢ}. The overlap between any of these signatures and the reference signature is preferably defined as

$${OL}_{i}^{ref} = \frac{\vert {S}_{i} ⋂ {S}_{ref} \vert}{\sqrt{\vert {S}_{i} \vert \cdot \vert {S}_{ref} \vert}} ,$$

where |...| refers to the size of a set and ∩ denotes intersection. All signatures Sᵢ whose overlap with the reference signature exceeds a certain threshold are included in the ‘recurrent signatures set’

$$R = \{ {S}_{i} \text{:} {OL}_{i}^{ref} > {t}_{rec} \} .$$

The threshold level t_{rec} has to be chosen large enough to discriminate against random fluctuations, but sufficiently small to include a significant fraction of signatures. In general t_{rec}=70% is used for the experiments below, but the results are robust with respect to the exact choice of this parameter. Finally a module is obtained by selecting only those genes that appear in at least a fraction f_{g} of all signatures in R. All genes within a module are assigned a score according to the average of their gene scores in all the signatures in R. The module conditions are defined correspondingly.

As described in further detail below, the present invention may also optionally be used for fusion of signatures in the global analysis. Pairs of recurrent signatures {Sᵢ, Sⱼ} are used as explained in the text. For each pair, the intersect Pᵢⱼ=Sᵢ∩Sⱼ of genes appearing in both signatures is computed as well as the overlap OLᵢⱼ between the signatures (similarly defined as OLᵢ). In order to construct modules the following method is preferably performed. First, the pair signature

$${P}_{ij}^{ref}$$

with the largest associated overlap

$${OL}_{ij}^{ref}$$

is selected as the ‘seed’ of a new module. Then all pair signatures Pᵢⱼ, whose overlap with

$${P}_{ij}^{ref}$$

exceeds a certain fraction t_{rec} of OLᵢⱼ^{ref} are assigned to the recurrent signatures set Rₖ, i.e.

$${R}_{k} = \{ {P}_{ij} \text{:} OL ( {P}_{ij} , {P}_{ij}^{ref} ) > {t}_{rec} {OL}_{ij}^{ref} \} .$$

The gene content and scores of the associated module are obtained from Rₖ as described in the previous paragraph. Subsequently the pairs that have been assigned to Rₖ are removed from the total ‘pool’ of pair signatures {Pᵢⱼ}. In order to avoid the identification of further, less coherent realizations of the same module, those pairs from {Pᵢⱼ} are also removed which would have been assigned to Rₖ for a somewhat lower value of the threshold t_{rec}, unless they have a significant (˜75%) overlap with any other pair signature. This process is preferably iterated until all sets are assigned.

According to further preferred embodiments of the present invention, subsequently, the genes that exhibit an expression pattern similar to that of the input genes over this subset of highly scored conditions, are assigned to the module.

Modules defined by the method of the present invention are preferably considered reliable only if they are repetitively predicted by distinct input sets. This recurrence property is strongly preferred in order to exclude modules which do not reflect underlying regulation, but were obtained due to limited statistics.

The signature algorithm can also be applied iteratively by using the output gene-signatures as new input sets. Indeed, such an iterative application, starting from random input sets, converged to many yeast modules identified by the present analysis. Therefore, the iterative application of the signature algorithm is an optional but preferred embodiment of the present invention, although more preferably, this iterative application is combined with the recurrence method, as described in greater detail below.

The utility of the method of the present invention was assessed by applying it to data from the yeast S. cerevisiae, as described in greater detail below with regard to Examples 1-2. The results of this application demonstrate that when a cis-regulatory element common to a set of functionally linked genes is known, gene expression data can be used to extract the associated transcriptional module with the method of the present invention. Example 7 describes the application of the method of the present invention on computer-generated data.

The present invention may be more clearly understood with regard to the illustrations and accompanying description.

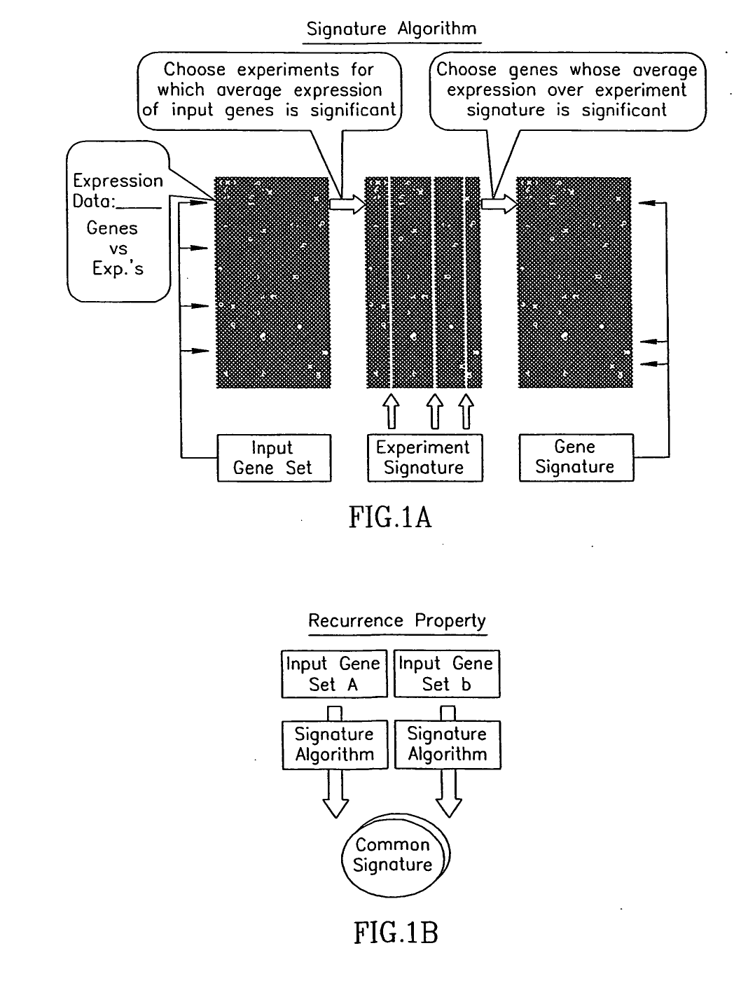

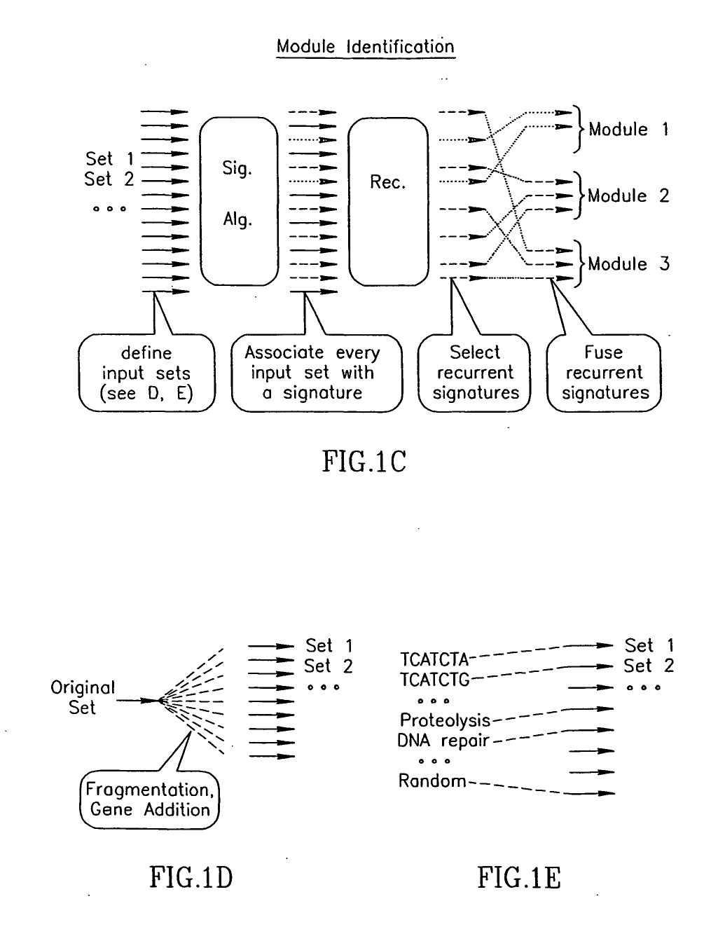

As previously described, a transcriptional module is more preferably specified by a two dimensional signature, which includes the co-expressed genes and the subset of relevant experimental conditions. Module signatures are optionally and preferably determined with respect to input sets of genes that comprise genes with similar functions, or genes that display a common regulatory motif in their upstream region (FIG. 1A). Similar function does not necessarily imply co-regulation and not all the genes displaying a regulatory motif are regulated by the corresponding transcription factor.

However, many such input sets may optionally include a core subset of co-regulated genes (2, 5, 11, 12, 13). The method of the present invention preferably uses gene expression data to create an experimental signature, as previously defined, and therefore to identify the transcriptional module corresponding to that core.

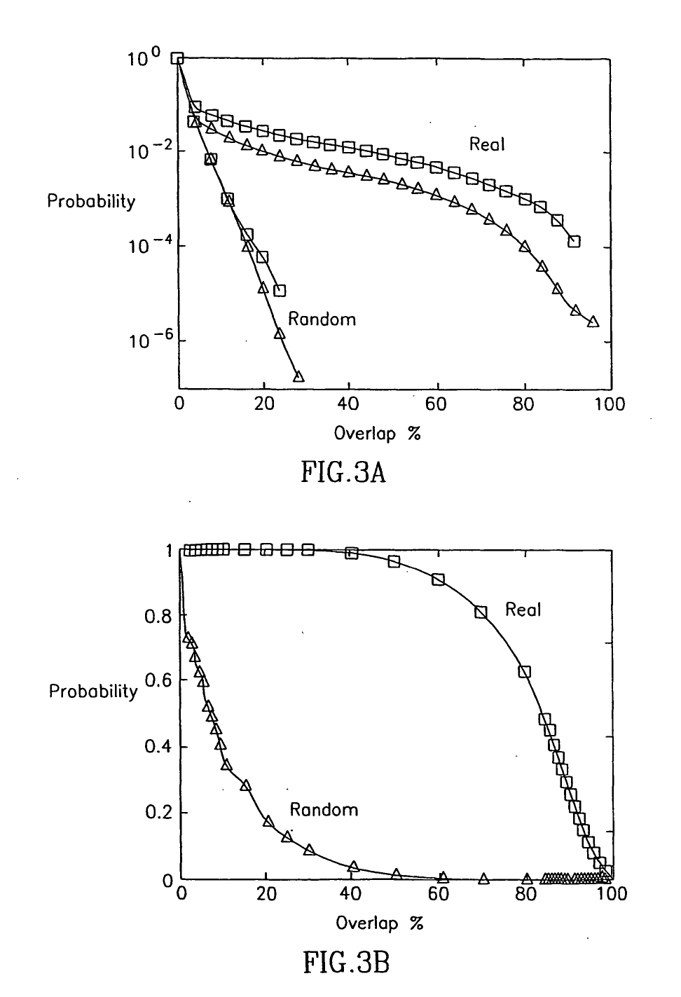

According to a specific, preferred implementation of the present invention, modules defined by the method of the present invention are preferably considered reliable only if they are repetitively predicted by distinct input sets. This recurrence property is strongly preferred in order to exclude modules which do not reflect underlying regulation, but were obtained due to limited statistics. The present invention optionally and more preferably features a reliability criterion based on the remarkable noise-resistance of the signature algorithm explained above, which allows the identification of the same co-regulated group of genes starting from numerous distinct input sets. Specifically, a signature is preferably considered to be reliable if it is obtained from several distinct input sets. For example, most input sets generated by the addition of random genes to a set of co-regulated genes give rise to the same gene-signature. In contrast, very different gene-signatures are obtained when the same procedure is applied to a group of genes that is not co-regulated (FIG. 3B). Indeed, the probability that two distinct input sets define similar modules is reduced to a very low number, or even vanishes, when randomized data, obtained by shuffling all components of the gene expression matrix, is used (FIG. 3A).

### EXAMPLE 1

### Analysis of Data From Yeast

The utility of the method of the present invention was assessed by applying it to data from the yeast S. cerevisiae. The following analysis uses publicly available data consisting of 850 genome-wide expression measurements, including the recent compendium of 300 measurements (15). The full list of references is given in the Supplementary Information. Functional classifications are according to the Munich Information Center for Protein Sequences (MIPS)(16).

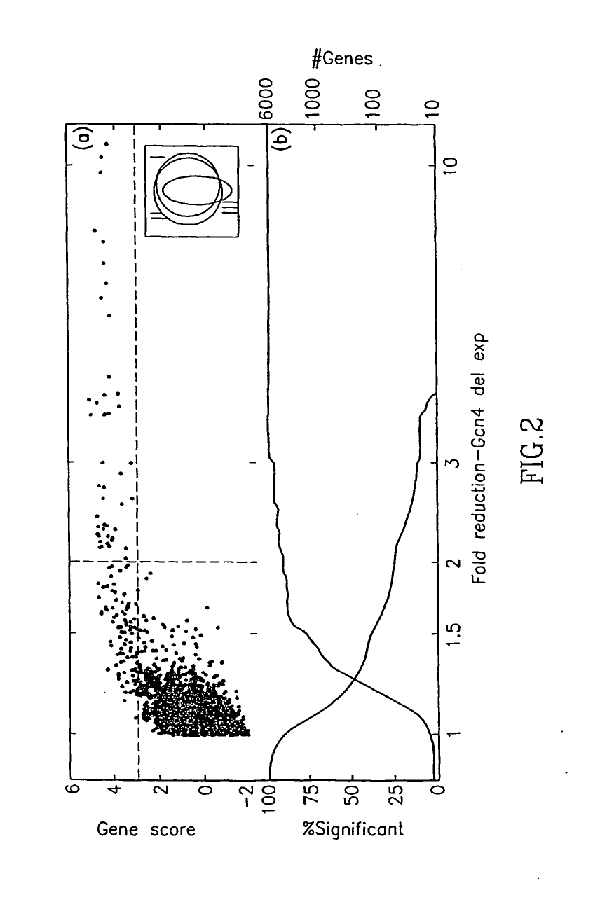

For the purposes of this example, the method of the present invention was used to identify the transcriptional module associated with the biosynthesis of amino acids. Two input sets related to this pathway were considered. The first input set was composed of the 119 MIPS classified amino-acid biosynthesis genes. The second input set included the 972 genes (˜15% of the yeast genome) that display in their upstream region the consensus-binding site for GCN4, the transcription factor that mediates the general response to amino acid starvation (17). Strikingly, both input sets define virtually the same transcriptional module (inset of FIG. 2A).

The computationally identified module is highly consistent with the results of a recent gene microarray experiment, which compared genome-wide expression levels of a GCN4-deletion strain with those of a wild type strain (15) (although this experiment was excluded from the computational analysis). Forty-six of the fifty-one genes whose expression in the GCN4 deletion strain was reduced by at least two-fold, were identified computationally. Moreover, the computational module includes most of the genes that were repressed more than ˜1.5 fold (FIGS. 2A, 2B). This consistency is particularly rewarding, as only 89 of the 972 genes composing the GCN4-related input set were also part of the transcriptional module. Thus, a small core subset of co-regulated genes is sufficient for extracting the full transcriptional module, even when the input set includes numerous non-relevant genes.

By contrast, a significantly lower fraction of the GCN4 regulated genes were grouped together by a hierarchical clustering algorithm which is known in the art (3) (data not shown). Notably, when the dataset was limited to the subset of relevant conditions composing the experimental signature of the method of the present invention, thereby using the added information gained by using the parameters of the present invention, a considerably better grouping was obtained (FIG. 3B, triangles). This result emphasizes the importance of associating a regulatory module with the relevant subset of conditions, rather than searching for correlated expression in all available data, and may demonstrate one reason for the greater efficacy of the method of the present invention. However, the method of the present invention is also clearly more effective because it uses this additional information in combination with an analysis which is designed to find connections between groups of genes, unlike background art methods.

The Recurrent Signature method of the present invention uses a large set of gene expression data to identify transcriptional modules and thus overcomes the experimental noise inherent in any single measurement. The transcriptional module associated with phosphate metabolism illustrates this point. Similar transcriptional modules (72% overlaps) were defined by two practically distinct input sets, the first of which was composed of the MIPS-classified phosphate metabolism genes, while the second included the genes which display the Pho4 binding site in their upstream region. This module is consistent with recent results of eight “wet bench” microarray experiments designed to explore the same module (18). In particular, four previously uncharacterized genes were assigned a role in phosphate metabolism following the microarray experiments. All four genes were retrieved computationally, thereby demonstrating the ability of the method of the present invention to perform a functional assignment of uncharacterized genes. Indeed, the module discovered by the method of the present invention displays a markedly reduced level of noise compared to individual experimental measurements, as is demonstrated in FIG. 2.

### EXAMPLE 2

### Further Genetic Data From Yeast

The method of the present invention was further applied to additional types of data from yeast, as examples of the previously described embodiments of the method of the present invention.

Section 1: Operation with Complete Information

As previously described, the method of the present invention is preferably used with genomic sequence data, transcriptional data, information about cis-regulatory elements and functional links between genes.

Section 2: Operation without Prior Knowledge of cis-regulatory Elements

However, even if only functional data and genomic sequences are available, the method of the present invention can still optionally be used to identify the transcriptional modules and optionally also the cis-regulatory elements. Examples of these different embodiments of the method of the present invention are described with regard to particular types of data in the following sections.

One hundred and thirty-three function-related input sets obtained from yeast, containing at least five genes, were defined according to the functional classifications scheme in the MIPS database (http://www.mips.biochem.mpg.de/proj/yeast/catalogues/funcat/index.html). 8192 sequence-related input gene sets were composed of genes displaying a specific oligomer of length seven in their 600 bp upstream region. Pairs of reverse complements were assumed to be equivalent. The method of the present invention was applied to each input set, defining function-related and sequence-related modules. Overlap between modules was defined as the number of common genes, normalized by the geometric mean of module sizes. Sequences were associated with a function when the overlap between the related modules was >60%. In the neighbor analysis, 700 sequences displaying an overlap >70% with at least one of their neighbors were clustered into groups of sequences with similar modules.

Indeed, systematic computation of the overlaps between all function-related and sequence-related modules (considering all possible oligomers of length seven) revealed that high overlaps are obtained in very specific cases (data not shown). Thus, for example, only fifteen of the sequence-related modules (0.2%) displayed an overlap higher than 70% with the amino-acid biosynthesis module, with most of these motifs representing minor deviations from the GCN4 consensus-binding site.

As previously described, the method of the present invention is still operative to identify the transcriptional modules, even if the cis-regulatory elements are not known. The method of the present invention can also optionally distinguish the cis-regulatory elements.

In the experiments of Section 1 above, it was noted that several sequences were associated at high overlap with each specific function-related module. In this Section, a technical aspect of the method of the present invention is described, for producing the consensus motif corresponding to these sequences in matrix form. The method was performed as follows. Consensus sequences were defined with respect to a particular group of sequences with overlapping modules. Similarity between two sequences was defined as the number of matching nucleotides at the optimal alignment. The sequence with the highest average similarity with all other sequences was chosen as a seed. This seed was used a starting point for defining a set of similar sequences by repeatedly adding to the set the sequence which is most similar to those sequences which are already in the set (provided that this similarity is greater than about 5). The motif that displayed the highest overlap with all other sequences in this set defines a ‘center’. The other motifs are aligned with the center, yielding the matrix of nucleotide distribution and defining the consensus. The sequence displaying the minimum similarity with the preceding seeds was chosen as a new seed and the process was repeated.

This method resulted in the identification of a group of coordinately regulated genes, as shown in Table 1, along with the cis-regulatory elements themselves. Remarkably, many of the cis-regulatory elements known to be involved in the regulation of the specific function were identified through the application of the above method (Table 1). Most of these motifs are not amongst the strongest over-represented sequences in the MIPS-classified input set, although some were identified by further statistical analysis of the upstream regions of the genes in this set (21, 22). Thus, the present invention is also able to identify novel cis-regulatory elements.

Section 3: Operation without Functional Information

As previously described, the method of the present invention is still operative to identify the transcriptional modules, even if functional information and/or linkages between the genes in these modules are not known. The method of the present invention can also optionally detect the cis-regulatory elements using genome wide expression data and genomic sequences. In the experiments of Section 2 above, it was noted that function-related cis-regulatory elements could be successfully identified through the application of the method of the present invention, which provided the impetus to explore the applicability of the method for identifying putative cis-regulatory elements together with the module of regulated genes, in cases where functional classifications are not available. To this end, the fact that most transcription factors bind to sites that represent a minor deviation from the consensus sequence was exploited in order to focus the application of the method.

In particular, the analysis focused on neighbor DNA motifs, namely two motifs that differ by a single nucleotide. Typically, the two input sets corresponding to neighbor motifs are essentially distinct. If, however, these motifs code for binding sites of the same transcription factor, they should define identical transcriptional modules. Indeed, many pairs of neighbor motifs define highly similar modules (FIG. 3A). Thus, according to the present invention, cis-regulatory element, optionally and preferably with the associated regulated genes, are identified by detecting a plurality of neighboring sequence motifs defining highly similar transcriptional modules

In sharp contrast, when randomized data is used, no neighbors were associated with an overlap higher than ˜20%. The probability that a pair of motifs will be associated with high overlap is significantly higher when these motifs are neighbors (FIG. 3A). Seven hundred motifs were identified which had a high overlap (>70%) with at least one of their neighbors. These sequences were further analyzed to identify putative motifs, the most significant of which are summarized in Table 2(19, 20). All known motifs presented in Table 1 were also recovered, and 21 out of the 46 consensus defined in the SCPD database were identified as significant in a similar analysis.

Importantly, in contrast with most existing methods for discovering cis-regulatory elements (21, 23, 24, 25, 26), the integrated approach of the method of present invention can identify the group of regulated genes together with the associated motif. Comparative analysis of the upstream regions of these genes should lead to a better understanding of the requirements for regulation by the specific motif. For example, the method of the present invention was used to determine that genes in most transcriptional modules exhibit the associated motif in close proximity to a preferred position (Tables 1-2). This positional bias is specific to the module, and provides a strong independent support for the validity of these results.

As demonstrated by the previously described results, when a cis-regulatory element common to a set of functionally linked genes is known, gene expression data can be used to extract the associated transcriptional module. The current Example explores the ability to identify the common cis-regulatory elements themselves, when these elements are not provided. The present approach relies on the expectation that a transcriptional module can be repetitively identified by the method of the present invention, which would identify this group of genes as a function-related module when applied to the corresponding MIPS-classified input gene set. The same module should also be recovered as a sequence-related module, starting from the input set of genes which exhibit the appropriate cis-regulatory element in their upstream region.

### EXAMPLE 3

### Pathway Refinement

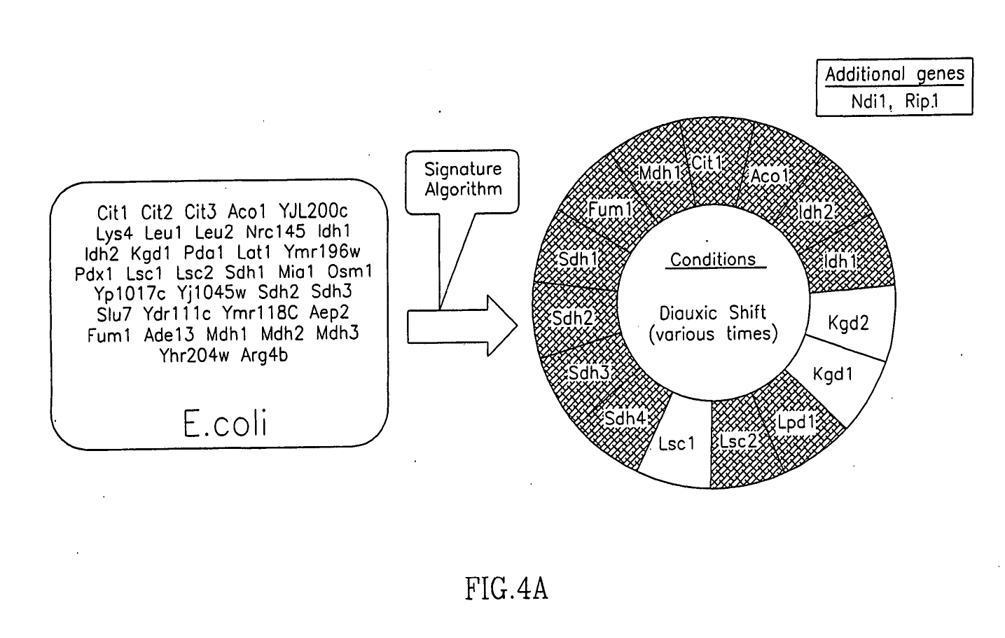

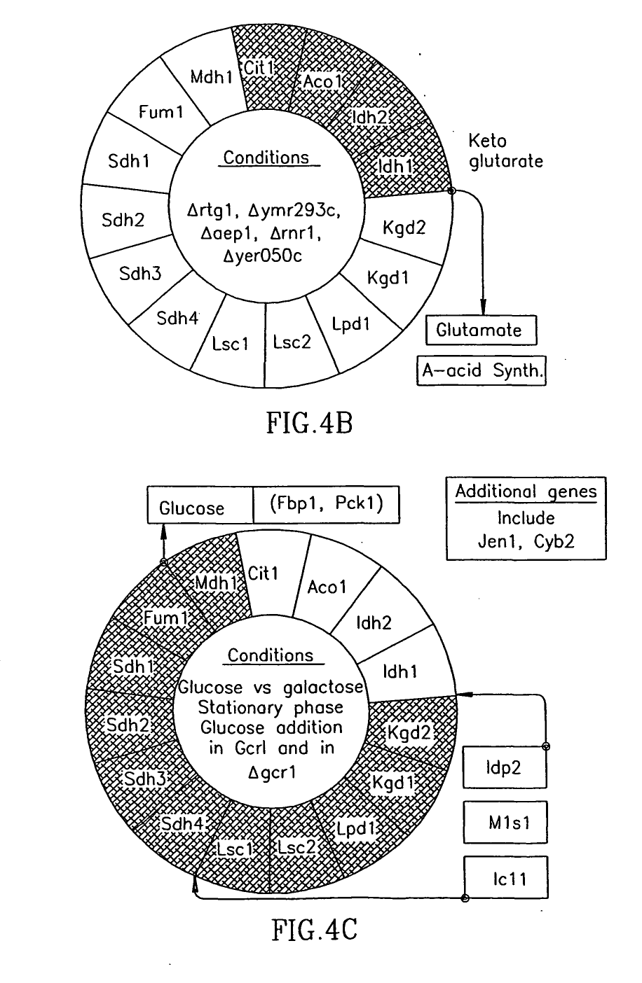

The method of the present invention may optionally and more preferably be used to elucidate the regulatory properties of a specific pathway. In this case, an initial input set is defined by genes that are presumed to be involved in the pathway. A transcriptional module can be identified from this initial set using the general scheme described above. For example, applying this approach to a homology-based guess for the TCA cycle genes in S. cerevisiae yields most of the genes that are indeed involved in this pathway, indicating that the TCA cycle genes are co-regulated at the transcriptional level (FIG. 4A). The method of the present invention also identifies two subparts of the cycle as autonomously co-regulated in different cellular contexts (FIGS. 4B and 4C).

According to the present analysis the genes upstream of α-KG, a primary precursor for glutamate, are co-regulated under conditions of rtg1-deletion and deletions affecting mitochondria function. Indeed, it was recently demonstrated that the expression of those genes becomes rtg1-dependent when mitochondria respiration capacity is compensated. Interestingly, under a different set of conditions, the genes upstream of α-KG appear to be co-expressed with cat8-dependent genes, suggesting their involvement in gluconeogenesis (FIG. 4C). This example illustrates the capability of the method of the present invention to identify context-dependent co-regulation with high resolution.

### EXAMPLE 4

### Global Study of the Yeast Transcriptional Modules

Describing the transcriptional program in terms of modules rather than genes provides a significant reduction of complexity and offers a global perspective on the organization of transcription. As discussed above, the recurrent signature approach is an ideal tool for this task, because a sufficiently diverse choice of input sets suffices to reveal the underlying modular structure. The computational efficiency of the signature algorithm allows its application to a large number of input sets. One possibility is to iteratively refine a large number of randomly chosen input sets (see below). An alternative approach is described in this Example, where different types of biological information are integrated into the analysis through the definition of the input sets.

Three classes of input sets were considered (FIG. 1E). First, the available genomic sequence was used to define sequence-related input sets. All possible combinations of six, seven and eight nucleotides were considered, corresponding to 4⁶, 4⁷ and 4⁸ possible sequences, respectively. A sequence related input set includes the genes that display the particular sequence in their upstream region. In cases where a sequence indeed functions as a cis-regulatory element, a subset of these genes is expected to be co-regulated by the associated transcription factor. Second, function-related input sets were defined according to the classification in the MIPS database¹⁴. Third, genes were clustered according to their expression profile using the previously described hierarchical clustering algorithm. The clusters emerging at tree levels 9 and 10 (typically 20 genes) were used as input sets. The signature algorithm was applied to all the above-mentioned input sets. The algorithm was coded in Matlab and the entire analysis was performed in less than three hours on a desktop PC computer.

The recurrence property was used to distinguish the reliable signatures. First, a search was performed for pairs of overlapping signatures that were generated from sequences that differ by a single nucleotide or are inverse complements of each other. Since the two input sets associated with each of those pairs are essentially distinct, coinciding signatures are generated only when both sequences bind the same transcription factor. This overlap requirement is important to distinguish the sequences that are involved in the regulation of a module from those that are merely over-represented.

Similarly, a search was performed for coinciding pairs of function-related or cluster-related signatures. These pairs of recurrent signatures were fused into transcriptional modules. The fusion algorithm resembles agglomerative clustering procedures, however the objects to be clustered are signatures rather than genes or conditions. In a subsequent part of the analysis, the modules were refined as is described in FIG. 1D (similar to the pathway analysis described above).

### EXAMPLE 5

### Higher Order Correlations in the Transcription Program

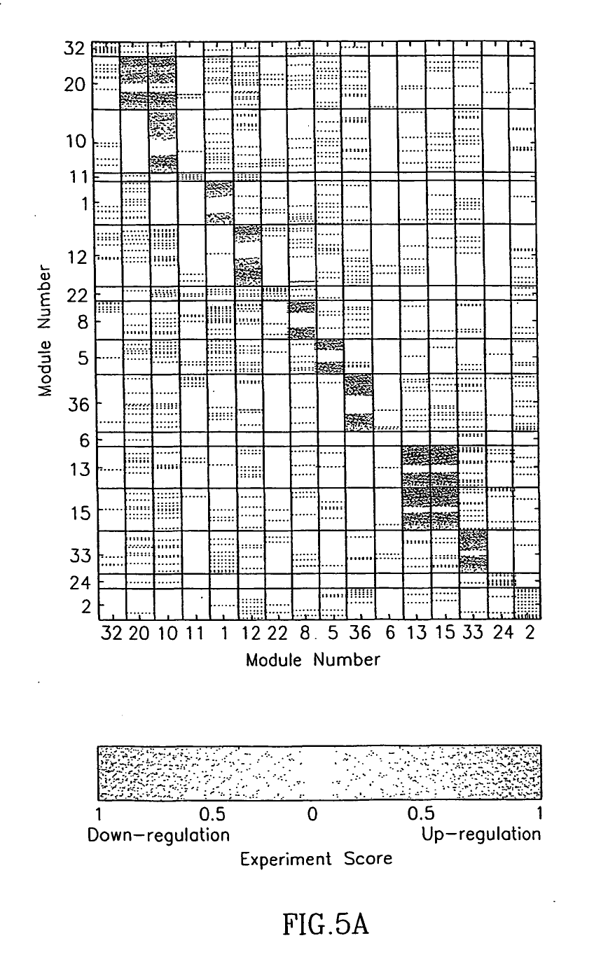

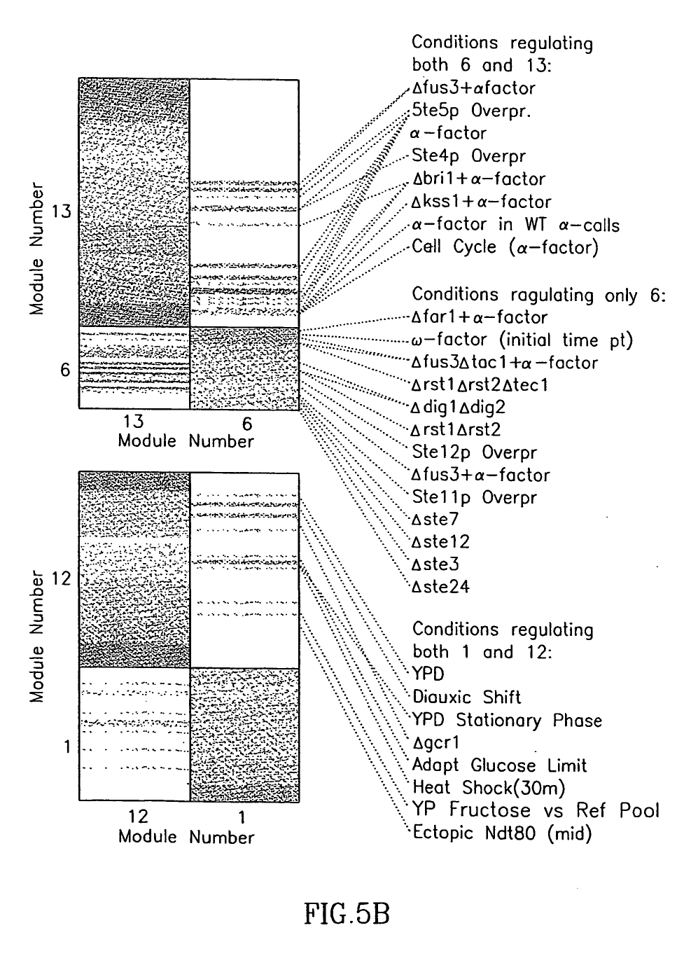

In general the experimental signatures of different modules may contain common experiments. Nevertheless each module is characterized uniquely by its experiment-signature and the associated score distribution, which contain valuable information about the function of the module. In particular, the experiment-signature can be used to reveal higher order correlations between different transcriptional modules. In order to identify such correlations the experiment-signature of a reference module was considered and the scores allotted to these experiments in the other modules were plotted (FIG. 5; see Table 1 for information). The number of lines indicates the number of experiments that each module shares with the reference module, while the scores of these experiments are represented by color-coding. For example, the experiment-signatures of module #10 (associated with rRNA processing) and module #20 (related to stress response) nearly coincide, but have reciprocal experimental scores. This strong inverse correlation indicates that rRNA processing is repressed under most stress conditions.

The module that is associated with the ribosomal proteins (#1) is distinct from module #10, although their experiment-signatures include mostly the same experiments with similar scores. The mitochondrial ribosomal proteins form yet another distinct module (#12), which is in fact induced by some perturbations that repress the ribosomal proteins. Note also that most conditions that induce the mating genes (#6) repress the module involved in the G1/S transition during the cell cycle (#13), reflecting the G1 arrest accompanying the mating response.

The signature algorithm can also be applied iteratively by using the output gene-signatures as new input sets. Indeed, such an iterative application, starting from random input sets, converged to many yeast modules identified by the present analysis. However, some modules were not revealed and even turned out to be unstable when used as input sets for subsequent iterations. An example is module #5, which is composed almost exclusively of genes that are involved in oxidative phosphorylation. The existence of such modules underlines the need for the recurrence requirement to obtain a complete picture of the transcriptional modules.

In addition to the identification of high order correlations in the transcriptional network, the collection of a large dataset of modules based on all available expression profiles can be used to facilitate the interpretation of the results of new microarray experiments. For a small set of experiments, the measured expression of individual genes is generally subject to a large amount of noise, such that only very significant changes in the expression permit a meaningful interpretation. However, analyzing such small-scale experiments in the context of the global analysis provides additional information, since it is not sensitive to the noise inherent in the expression measurement of a single gene but to the features of the data at the modular level. Thus this framework can serve as a platform for analyzing novel microarray experiments.

### EXAMPLE 6

### Functional Coherence of Modules

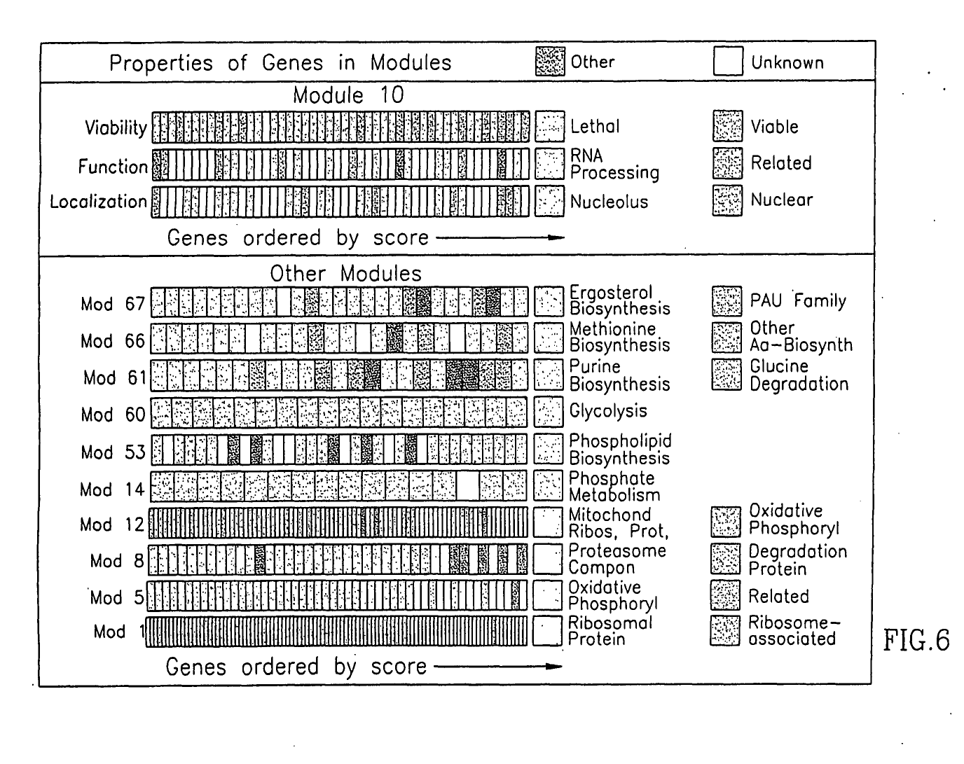

The functional coherence of the modules is illustrated in FIG. 6 (see Table 1 for information). As shown in FIG. 6, in most modules, the genes of known function are almost exclusively involved in one particular cellular process. As a consequence functional links can be assigned to the genes of unknown function in each of the modules. The functional coherence of the known genes suggests the reliability of these links. For example, one of the most reliable modules which was identified (module #10, as shown in the top portion of FIG. 6) consists mostly of essential genes of unknown function, while most known genes are involved in rRNA processing. The fact that all genes in this module with known cellular localization are found in the nucleus, with the majority in the nucleolus, further supports the association of these genes with rRNA processing. The method of the present invention reveals a large variety of modules using all available experiments, distinguishes the reliable ones, and provides an accurate definition of their gene content and regulatory context.

### EXAMPLE 7

### Analysis of Computer-Generated Data

The ability of the method of the present invention to identify overlapping transcriptional modules is further demonstrated by using computer-generated expression data as a model system. A model consisting of 1050 genes and 30 transcription factors (TF) was considered. The regulation of each gene was specified by a promoter matrix P, with pᵢⱼ={−1, 0, 1} specifying that TF aⱼ represses, does not affect or activates gene i, respectively. Of the total genes regulated by each TF, 80% (20%) were activated (repressed). The log expression of gene i at condition μ was defined as

$${g}_{i}^{μ} = log ( {\prod }_{j = 1}^{30} exp ( {p}_{ij} {t}_{j}^{μ} ) )$$

where tⱼ^{μ}={0,1} specifying the activity of TF j at condition μ. An average of 6 randomly chosen TFs were active at each condition. An expression matrix consisting of 4000 conditions was used in the analysis.

The ‘transcriptional modules’ in the model-network correspond to the group of genes that are regulated by each of the underlying transcription factors. The data was both clustered using a background art hierarchical clustering algorithm and was also analyzed by using the method of the present invention.

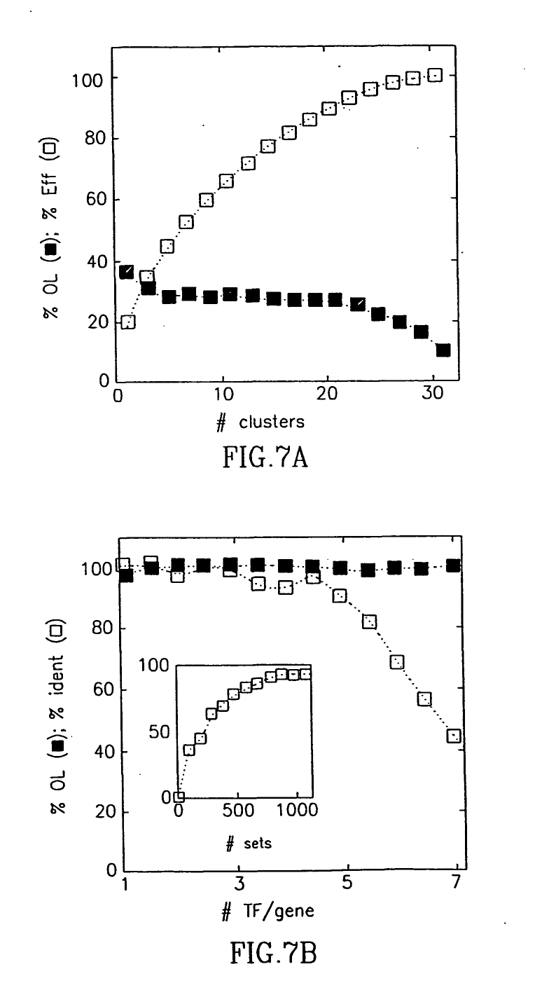

When a gene is regulated by a single factor, clustering identifies all of the underlying modules. Combinatorial regulation, however, severely impairs the clustering results (FIG. 7A). The Recurrent Signature method of the present invention, on the other hand, recovers the modules corresponding to most transcription factors in the system (FIG. 7B). This was accomplished by using gene clusters as the input sets to the method of the present invention. To enhance the number of input sets used by the Recurrence Signature method of the present invention, 15 groups of 500 randomly chosen conditions were clustered, resulting in ˜1000 different input groups. In the application of the method of the present invention, the condition threshold was taken with n=3, while the gene threshold was one standard deviation. Modules were considered reliable if they were recovered at least 3 times, with an overlap of 95%. A gene was assigned to the final module if it was part of at least two such overlapping modules.

Importantly, all computationally identified modules are practically identical to the underlying modules. For a limited number of input sets and high combinatorial level, not all of the modules are identified, reflecting the insufficient statistics (FIG. 7B, inset).

Conclusions

The reliability of a transcriptional module, as determined by the method of the present invention, can optionally be measured by the number of its recurrences and the overlaps between them. Similarly, the reliability of assigning a specific gene to the module can be estimated by its re-appearance in the different recurrences. In contrast, most clustering algorithms which are known in the background art lack such objective measures of reliability. Note also that according to the method of the present invention, modules are identified as distinct only if the overlap between them is lower than the error in the identification of any single module. As more data, scanning a larger region of possible conditions, becomes available, more overlapping modules can be identified. Importantly, within the Recurrent Signature approach of the method of the present invention, and in contrast to most clustering algorithms which are known in the background art, the addition of experiments to the database does not impair the identification of any specific module, since non-relevant conditions are automatically be excluded from the analysis.

Without wishing to be limited to one or more particular applications of the method of the present invention, clearly the present invention has a large number of advantages over background art methods for uncovering functional relationships between groups of genes, particularly those genes which are regulated in a coordinate manner. These advantages suggest further possible applications of the method of the present invention, non-limiting examples of which are described below in greater detail. In addition, the description below also summarizes some of the main features of the method of the present invention.

Decomposing the genome into modules of co-regulated genes provides the first essential step in ‘reverse engineering’ the transcriptional networks by simplifying their complexity and revealing higher order structures. This method is conceptually simple, fast and efficient. It can effectively be used not only when both expression profiles and functional classifications are known, but also when only expression data and genomic sequence are provided. Further applications of the method of the present invention include, but are not limited to, studying the design principles underlying the global transcriptional program. As more data will become available through studies of gene transcription and the regulation thereof, the method of the present invention can also be used to study the transcriptional networks underlying gene regulation in higher eukaryotes.

While the invention has been described with respect to a limited number of embodiments, it will be appreciated that many variations, modifications and other applications of the invention may be made.

### REFERENCES

1. L. H. Hartwell, J. J. Hopfield, S. Leibler, A. W. Murray, Nature 402, C47-52 (1999).

2. M. B. Eisen, P. T. Spellman, P. O. Brown, D. Botstein, Proc Natl Acad Sci USA 95, 14863-14868 (1998).

3. U. Alon, et al., Proc Natl Acad Sci USA 96, 6745-6750 (1999).

4. P. Tamayo, et al., Proc Natl Acad Sci USA 96, 2907-2912 (1999).

5. M. P. Brown, et al., Proc Natl Acad Sci USA 97, 262-267 (2000).

6. C. H. Chu, H. Bolouri, E. H. Davidson, Science 279, 1896-1902 (1998).

7. K. Struhl, D. Kadosh, M. Keaveney, L. Kuras, Z. Moqtaderi, Cold Spring Harb Symp Quant Biol 63, 413-421 (1998).

8. M. A. Simon, Cell 103, 13-15 (2000).

9. Y. Cheng, G. M. Church, Ismb 8, 93-103 (2000).

10. G. Getz, E. Levine, E. Domany, Proc Natl Acad Sci USA 97, 12079-12084 (2000).

11. F. P. Roth, J. D. Hughes, P. W. Estep, G. M. Church, Nat. Biotech 16, 939-945 (1998).

12. S. Tavazoie, J. D. Hughes, M. J. Campbell, R. J. Cho, G. M. Church, Nat Genet 22, 281-285 (1999).

13. C. Niehrs, N. Pollet, Nature 402, 483-487 (1999).

15. T. R. Hughes, et al., Cell 102, 109-126 (2000).

16. H. W. Mewes, K. Albermann, K. Heumann, S. Liebl, F. Pfeiffer, Nucleic Acids Res. 25, 28-30 (1997).

17. A. G. Hinnebusch, in The molecular and cellular biology of the yeast Saccharomyces J. R. Broach, J. Pringle, E. W. Jones, Eds. (Cold Spring Harbor Press, Cold Spring Harbor, 1992), vol. 2, pp. 319-414.

18. N. Ogawa, J. DeRisi, P. O. Brown, Mol Biol Cell 11, 4309-4321 (2000).

21. J. van Helden, B. Andre, J. Collado-Vides, J Mol. Biol. 281, 827-842 (1998).

22. J. D. Hughes, P. W. Estep, S. Tavazoie, G. M. Church, J Mol Biol 296, 1205-1214 (2000).

23. G. D. Stormo, G. W. Hartzell, Proc. Natl. Acad. Sci. 86, 1183-1187 (1989).

24. G. Z. Hertz, G. W. Hartzell, G. D. Stormo, Comput Appl Biosci 6, 81-92 (1990).

25. E. Moskvina, C. Schuller, C. T. Maurer, W. H. Mager, H. Ruis, Yeast 14, 1041-1050 (1998).

26. H. J. Bussemaker, H. Li, E. D. Siggia, Proc Natl Acad Sci USA 97, 10096-10100 (2000).

28. P. D. Gregory, A. Schmid, M. Zavari, M. Munsterkotter, W. Horz, EMBO J 18, 6407-6414 (1999).

What is claimed is:

1. A method for analyzing experimental data obtained from observing biological processes, at least two of the biological processes being regulated in a coordinate manner, comprising:
analyzing the experimental data to identify at least two biological processes undergoing a significant, recurrent change under similar experimental conditions; and
identifying at least one functional linkage between the biological processes according to said analysis of the experimental data.

2. The method of claim 1, wherein the biological process is the behavior of groups of coordinately regulated genes.

3. The method of claim 2, wherein the experimental data includes large-scale gene expression data and genomic sequence.

4. The method of claim 3, wherein each group of coordinately regulated genes forms a transcriptional module specified by a signature, said signature including data from said coordinately regulated genes and relevant experimental conditions.

5. The method of claim 4, wherein said transcriptional module is determined with respect to input sets of genes having a functional link.

6. The method of claim 5, wherein said functional link is selected from the group consisting of similar functions for said genes, or displaying a common regulatory motif in the upstream region of said genes.

7. The method of claim 6, wherein said transcriptional module is also determined using genomic sequence.

8. The method of any of claims 2-7, wherein at least one cis-regulatory element for regulating said genes is also identified.

9. The method of any of claims 2-8, wherein a functional is determined to be reliable if said functional linkage is detected from a plurality of separate experimental data sets.

10. The method of claim 3, wherein each group of coordinately regulated genes forms a transcriptional module, said transcriptional module being identified according to said functional information, large scale gene expression data and genomic sequence.

11. The method of claim 10, wherein said transcriptional module is identified only according to said transcriptional data and genomic sequence.

12. The method of either of claims 10 or 11, wherein at least one cis-regulatory element for regulating said genes is also identified.

13. The method of claim 4, wherein said signature is created by:
scoring each experimental condition by the average (log) fold change in the expression data of the input genes at that specific experiment; and
retaining high scoring experimental conditions to form the experimental signature.

14. The method of claim 13, wherein each experimental condition is scored by the average (log) fold change in the expression of the input genes for a particular experiment.

15. The method of claim 14, wherein said transcriptional module is created by:
comparing expression data for additional genes to the expression data for the input genes according to the experimental signature; and
if said expression data for said additional genes is similar to the expression data for the input genes, assigning said additional genes to said transcriptional module.

16. The method of claim 15, wherein said input set of genes is I={i₁, . . . , i_{K}} and wherein identifying said transcriptional module includes:
identifying a plurality of components g^{μ}ᵢ of the gene expression matrix, said components being the log fold expression-change of gene i at experimental condition μ; i=1 . . . N and μ=1 . . . P, where N (P) denotes the total number of genes (conditions);
defining two normalized matrices
$$\{ {\hat{g}}_{i}^{μ} \} and \{ {\underline{g}}_{i}^{μ} \} ,$$
such that for every condition
$$μ , < {\hat{g}}_{i}^{μ} {>}_{i} = 0 , < {( {\hat{g}}_{i}^{μ} )}^{2} {>}_{i} = 1 ,$$
and for every gene i,
$$< {\underline{g}}_{i}^{μ} {>}_{μ} = 0 , < {( {\underline{g}}_{i}^{μ} )}^{2} {>}_{μ} = 1 ,$$
wherein the symbol < >ₓ denotes the average with respect to x;
scoring experimental conditions by the average change in the expression of the input genes
$${s}^{μ} = < {\hat{g}}_{i}^{μ} {>}_{i ∈ l} ;$$
defining an experimental signature M={μ₁, . . . , μ_{L}} with the highest scoring conditions, with
$$\vert {s}^{μ} \vert > n / \sqrt{KN}$$
where n=2 or 3;
scoring genes by
$${s}_{i} = < {s}^{μ} {\underline{g}}_{i}^{μ} {>}_{μ ∈ M} ;$$
and
including the highest scoring genes for which sᵢ>3σ, with σ the standard deviation of sᵢ, in said transcriptional module.

17. The method of any of claims 13-16, wherein a plurality of input sets of genes is used to define each transcriptional module, such that said transcriptional module is considered to be reliably identified only if said transcriptional module is predicted by a plurality of distinct input sets of genes.

18. The method of any of claims 2-17, wherein a cis-regulatory element is identified by detecting a plurality of neighboring sequence motifs for said genes.

19. The method of claim 1, wherein said functional linkage is a cis-regulatory element, such that said cis-regulatory element is identified.

20. The method of claim 19, wherein the biological process is the behavior of groups of coordinately regulated genes, and said genes regulated by said cis-regulatory element are identified with said cis-regulatory element.

21. The method of claim 1, wherein said functional linkage is an unidentified open reading frame, such that said open reading frame is identified.

22. Computerized code for analyzing experimental data obtained from observing biological processes, at least two of the biological processes being regulated in a coordinate manner, the code performing a method comprising:
receiving data from a plurality of experimental results;
scoring said experimental results according to a change in a behavior of a biological process;
scoring each biological process according to a change in said experimental results having at least a minimum score;
wherein said biological process is considered to be regulated in a coordinate manner with at least one other biological process if said biological processes receive a score above a minimum level.

23. The code of claim 22, wherein said biological process is performed by a gene, and said experimental results measure a change in expression of said gene.

24. The code of claims 22 or 23, wherein said minimum level is determined according to a statistically significant difference.

25. A method for analyzing experimental data obtained from observing biological processes, at least two of the biological processes being regulated in a coordinate manner, the method comprising:
receiving data from a plurality of experimental results;
scoring said experimental results according to a change in a behavior of a biological process;
scoring each biological process according to a change in said experimental results having at least a minimum score;
wherein said biological process is considered to be regulated in a coordinate manner with at least one other biological process if said biological processes receive a score above a minimum level.

26. The method of claim 25, wherein said biological process is performed by a gene, and said experimental results measure a change in expression of said gene.

27. The method of claims 25 or 26, wherein said minimum level is determined according to a statistically significant difference.

28. The method of any of claims 26 or 27, wherein an element E_{cg} of the gene expression matrix contains the log-fold expression-change of gene gεG={1, . . . , N_{g}} at the experimental condition cεC={1, . . . , N_{c}}, where N_{g} (N_{c}) denotes the total number of genes (conditions); two normalized expression matrices
$${\tilde{E}}_{c}^{g} {\hat{E}}_{g}^{c} and {\hat{E}}_{g}^{c} {\tilde{E}}_{c}^{g}$$
have zero mean and unit variance with respect to genes and conditions, respectively:
$${⟨ {\tilde{E}}_{c}^{g} ⟩}_{g ∈ G} = 0 , {⟨ {( {\tilde{E}}_{c}^{g} )}^{2} ⟩}_{g ∈ G} = 1 and$$
$${⟨ {\hat{E}}_{g}^{c} ⟩}_{c ∈ C} = 0 , {⟨ {( {\hat{E}}_{g}^{c} )}^{2} ⟩}_{c ∈ C} = 1 ,$$
where [symbol] ... [symbol] ₓ denotes the average with respect to x; an initial input set is a collection of N₁ genes: G₁={g₁, . . . , g_{N}_{₁}}⊂G; and
scoring said experimental results is performed according to an average change in gene expression within said input set, such that said experimental score is
$${s}_{c} = {⟨ {\tilde{E}}_{c}^{g} ⟩}_{g ∈ {G}_{1}}$$
and such that experiment-signature S_{c} contains said experimental results having a statistically significant absolute score.

29. The method of claim 28, wherein said genes are scored according to a weighted average change in an expression of each gene within said experiment-signature and wherein said gene score is
$${s}_{g} = {⟨ {s}_{c} {\hat{E}}_{g}^{c} ⟩}_{c ∈ {S}_{c}} .$$

30. The method of any of claims 25-30, wherein a plurality of biological processes are determined to be coordinately regulated according to a comparison between a reference set of a plurality of coordinately regulated biological processes and at least one additional biological process.

31. The method of claims 28-30, wherein said gene signature is determined for a reference input set
$${G}_{1}^{ref}$$
and a set of input sets
$$\{ {G}_{1}^{( i )} \}$$
obtained according to said experimental results, resulting in a reference signature S_{ref} and a collection of modified signatures {Sᵢ}, wherein said comparison is performed according to an equation:
$${OL}_{i}^{ref} = \frac{\vert {S}_{i} ⋂ {S}_{ref} \vert}{\sqrt{\vert {S}_{i} \vert \cdot \vert {S}_{ref} \vert}} ,$$
where |...| refers to the size of a set and ∩ denotes intersection.

32. The method of claim 31, wherein signatures Sᵢ having an overlap with said reference signature exceeding a threshold are included in a recurrent signatures set
$$R = \{ {S}_{i} : {OL}_{i}^{ref} > {t}_{rec} \} .$$

33. The method of claim 32, wherein said genes are determined to be coordinately regulated by selecting genes appearing in at least a fraction f_{g} of all signatures in R.

34. The method of any of claims 25-33, wherein said scoring of each biological process according to a change in said experimental results having at least a minimum score is performed more than once, such that said biological processes having said score above said minimum level are used as an input to said scoring again.
